(19) United States
(12) Patent Application Publication
(10) Pub. No.: US 2022/0133842 A1
(43) Pub. Date: May 5, 2022

(54) Use of fungal cyclic peptides of the destruxin type as antibacterial agents active against Clostridium perfringens

(71) Applicants: Centre national de la recherche scientifique, PARIS (FR); Université d'Aix-Marseille, MARSEILLE (FR)

(72) Inventors: Marc MARESCA, VITROLLES (FR); Josette PERRIER, MARTIGUES (FR); Cendrine NICOLETTI, SANARY SUR MER (FR); Sybille TACHON, MARSEILLE (FR); Clarisse ROBLIN, MARSEILLE (FR); Michael LAFOND, LE PRADET (FR); Hamza OLLEIK, MARSEILLE (FR); Michel FONS, AIX-EN-PROVENCE (FR)

(21) Appl. No.: 17/433,765

(22) PCT Filed: Feb. 25, 2020
(86) PCT No.: PCT/EP2020/054877

§ 371 Date: Aug. 25, 2021

(30) Foreign Application Priority Data
Feb. 25, 2019 (FR) FR1901896

(51) Int. Cl. A61K 38/12; A61K 36/062; A61K 38/15; A61P 31/04

(52) U.S. Cl. CPC A61K 38/12; A61P 31/04; A61K 38/15; A61K 36/062

(57) ABSTRACT

The present invention relates to the use of a destruxin or derivatives thereof to prevent or control Clostridium perfringens, in human or veterinary therapy, or in animal nutrition.

The present invention relates to the field of prevention or treatment of bacterial infections, especially by Clostridium perfringens.

Clostridium perfringens is responsible for intestinal infections in humans and animals. In humans, Clostridium perfringens is responsible for food poisoning. Clostridium perfringens is one of the most common causes of food poisoning in the United States and Canada (Johnson, E. A., Summanen, P., & Finegold, S. M. (2007). Clostridium. In P. R. Murray (Ed.), Manual of Clinical Microbiology (9th ed., pp. 889-910). Washington, D.C.: ASM Press). In France, Clostridium perfringens ranks 4th in terms of the number of outbreaks (2006-2007) and 1st (2006) or 3rd (2007) (Table 3) in terms of the number of cases among the causes identified in the mandatory declaration (DO) of collective food-borne illnesses (TIAC) (source ANSES, https://www.anses.fr/fr/system/files/MIC2010sa0235Fi.pdf). In farm animals, Clostridium perfringens mainly infects pigs and poultry with economic (reduced yield, mortality and treatment costs) and health (transmission to humans) consequences. In poultry farms, it can cause in its clinical form a very abnormally high mortality (up to 50% of the livestock) and in its sub-clinical form an infection with Clostridium perfringens usually results in significant economic losses since the yield of the animals is greatly reduced.

It is desirable to develop compositions that act specifically against this strain so as not to cause changes in the intestinal flora.

Destruxins are fungal cyclic peptides of the cyclohexadepsipeptide type produced by various fungi mainly of the genus Metarhizium anisopliae but also of the genera Metarhizium brunneum, Beauveria felina, Ophiocordyceps coccidiicola, Alternaria brassice, Alternaria linicola and Aschersonis sp.

There are various destruxins (35 molecules identified to date) grouped into 7 series (series A, B, C, D, E, F and G) (Pedras et al. Phytochemistry 2002, 59, 579-96).

They are formed by five amino acids (with spatial conformation S) and an alpha-hydroxy acid (with spatial conformation R). The destruxins of the various series differ by the type of amino acids, the type of alpha-hydroxy acid and/or the presence or absence of N-methylation of the amino acids. These molecules are known to have various biological activities (Wang et al., Molecules 2018, 23, 169; doi:10.3390) such as insecticidal, cytotoxic, immunosuppressive, antiproliferative, and antiviral activities (Pedras et al, Phytochemistry, 2002, 59, 579-96 and Wang et al., Molecules 2018, 23, 169; doi:10.3390) and some patent applications have allegedly claimed activity against osteoporosis (CN101433214B, CN106810601A, WO2002064155A1). However, their antibacterial effect has never been reported (Wang et al., Molecules 2018, 23, 169; doi:10.3390). The reported activity against Helicobacter pylori (Kao et al., Process. Biochem. 2015, 50, 134-139) does not correspond to an antibacterial activity but to an activity of inhibition of the vacuolisation caused by H pylori in gastric cells.

Unexpectedly, destruxins have now been shown to have activity against Clostridium perfringens. Moreover, unlike other fungal cyclic peptides (Enniatins A, A1, B, B1 and Beauvericin in particular, which have a broad spectrum of action with antibacterial activity on several Gram+ bacteria), the destruxins have shown selectivity of action against Clostridium perfringens. This selective activity of destruxins makes it possible to envisage their use for the treatment and/or prevention of infections linked to Clostridium perfringens, in particular intestinal infections in humans and farm animals, including chickens.

According to a first object, the present invention relates to a composition comprising at least one destruxin for treating and/or preventing Clostridium perfringens infections.

According to one embodiment, the destruxin is selected from destruxins of the fungus series A, B, C, D, E, F or G, or derivatives thereof.

“Destruxins” refers to cyclic peptides of the cyclohexadepsipeptide type, such as those produced by fungi of the genus Metarhizium anisopliae, Metarhizium brunneum, Beauveria felina, Ophiocordyceps coccidiicola, Alternaria brassice, Alternaria linicola and Aschersonis sp.

Examples include the A, B, C, D, E, F and G series of destruxins described by Pedras et al. Phytochemistry, 2002, 59, 579-96, including destruxins:
Series A: Dx A, A₁, A₂, A₃, A₄, A₅, A₄ chlorohydrin, desmethylDx A, dihydroDx A;
Series B: B, B₁, B₂, desmethylDx B, Desmethyl Dx B₂, homoDx, protoDx, hydroxyDx B, hydroxyhomoDx B, beta-D-Glucopyranosyl-hydroxyDx B;
Series C: C, C₂, desmethylDx C;
Series D: D, D₁, D₂
Series E: E, E₁, E₂, E chlorhydrin, E₂ chlorohydrin, E diol, E₁ diol;
Series F: F;
PseudoDx A, PseudoDx B.

According to a particular embodiment, we can mention in particular destruxins A, B, C, D, F or G and their derivatives (the sources of which are indicated in particular in Pedras et al, supra), in particular destruxins A and B. Destruxins A and B are commercially available (Sigma-Aldrich or A2S, purity >98%).

A destruxin according to the invention includes the above-mentioned destruxins, as well as their derivatives, in particular defined by the general formula (I) below.

The destruxin for the antibacterial application of the invention is a functional destruxin. “Functional destruxin” or “functionally active destruxin” means a destruxin with activity to prevent and/or treat a bacterial infection. Whether a protein is functional can be determined by any known method, for example by an in vitro assay for antibacterial activity (MIC, as described in Example 1).

The destruxin according to the invention may be of fungal or synthetic origin, preferably of fungal origin.

The term “fungal destruxin” includes fungal destruxin as defined above or a derivative thereof.

As destruxin according to the invention, we can mention in particular the compounds of formula (I):

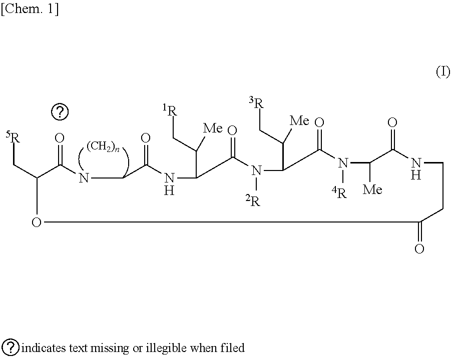

In which
¹R represents a hydrogen atom, a C1-C6 alkyl group or an aralkyl group;
²R, ³R, ⁴R, which may be identical or different, independently represent a hydrogen atom or a C1-C6 alkyl group;
⁵R represents a group selected from C2-C₆ alkenyl and C1-C6 alkyl groups optionally substituted by one or more substituents selected from halogen atoms, hydroxy (OH), carboxy (COOH), -glycosyl groups, and 3- to 6-membered heterocyclic groups comprising one or more heteroatoms selected from N, O and S;
   In particular:
¹R represents a hydrogen atom, a methyl group or a benzyl group;
²R, ³R, ⁴R, which may be identical or different, independently represent a hydrogen atom or a methyl group;
⁵R represents a group selected from CH═CH₂, —CHOHCH₂Cl, CH═CH₂, —CHMe₂, —COHMe₂, —C(O-beta-D-glycosyl)Me₂, —CHMeCH₂OH, —CHMeCOOH, [Chem2]

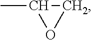

—CHOHCH₂Cl, —CHOHCH₂OH, —CHOHMe, —CHMe₂.

According to the present invention, Alkyl radicals represent straight or branched chain saturated hydrocarbon radicals of 1 to 6 carbon atoms, such as methyl, ethyl, propyl, butyl, pentyl, hexyl, isopropyl, tert-butyl, 2-methylbutyl, 2-methylpentyl, 1-methylpentyl. Halogen atoms include fluorine, chlorine, bromine and iodine, preferably fluorine.

Alkenyl radicals are straight- or branched-chain hydrocarbon radicals with 2 to 6 carbon atoms and comprising one or more ethylenic double bonds. Alkenyl radicals include allyl or vinyl radicals.

The term aralkyl refers to AlkylAryl groups where alkyl is defined as above and aryl refers to a mono- or bi-cyclic aromatic hydrocarbon system of 6 to 10 carbon atoms. Among the -AlkylAryl radicals, we can mention the benzyl or phenethyl radical.

The composition of the invention may comprise a destruxin in pure form in admixture, or in the form of a destruxin-producing fungus, or an extract thereof, such as a grind or culture supernatant thereof, including an extract comprising a destruxin, or mixtures thereof.

Wang et al. (supra) describes, among other things, different destruxins and the fungi that produce them (pages 14 and 15). Fungi producing a destruxin include Metarrhizium, Beauveria, Ophiocordyceps, Alternaria and Aschersoni and in particular the genera Metarrhizium anisopliae, Metarhizium brunneum, Beauveria felina, Ophiocordyceps coccidiicola, Alternaria brassicae, Alternaria linicola, Ophiocordyceps coccidiicola, Alternaria brassicae and Aschersonis sp; in particular Beauveria felina, Metarrhizium anisopliae, Metarhizium brunneum, Ophiocordyceps sp, Alternaria alternate, Alternaria brassicae, Alternaria linicola; and mixtures thereof, or extracts therefrom, and/or culture supernatants thereof.

Some of these genera are commercially available or available from depository organisations: Beauveria felina and Metarhizium anisopliae are commercially available from DSMZ and ATCC, e.g. DSM 4678 and ATCC® 60335™ or DSM 1490

Some of these genera are commercially available or available from depository organisations. They are commercially available from DSMZ and ATCC, for example Beauveria felina as DSM 4678; Metarrhizium anisopliae as ATCC® 60335™, DSM 1490 and DSM 21704; Metarhizium brunneum as ATCC® 90448™; Ophiocordyceps sp. as ATCC® 24400™; Alternaria alternate as ATCC® 13963, ATCC® 66981, DSM-12633, DSM-62006, DSM-62010 or DSM-1102; Alternaria brassicae as ATCC® 58169, ATCC® 38713 or ATCC® 34642; Alternaria linicola as ATCC® 201065, ATCC® 11802 or ATCC® 201658.

Culture supernatant or secretome means the culture medium in which the fungus has been grown, after separation of the fungus.

According to the present invention, the compounds of formula (I) exhibit specific antibacterial activity against Clostridium perfringens.

The compounds of formula (I) are therefore useful in the treatment and/or prevention of Clostridium perfringens-related infections.

The compositions according to the invention can be used in human or veterinary therapy to treat an infection caused by Clostridium perfringens, or as a feed supplement for animals to prevent infection by Clostridium perfringens.

Advantageously, and in contrast to antibiotics such as metronidazole traditionally used to treat Clostridium perfringens infections, the administration of destruxin does not induce the selection of resistant bacteria (see FIG. 1).

Clostridium perfringens comprises or consists of the ATCC®13124™ sequence deposited with the ATCC. For example, Clostridium perfringens may comprise or consist of a sequence having a degree of identity of at least 80% to said commercially available sequence ATCC®13124™, in particular at least 85% identity, preferably at least 90% identity, and more particularly at least 95%, at least 96%, at least 97%, at least 98% or at least 99% identity, with the proviso that said sequence of Clostridium perfringens is functional.

According to another object, the present invention thus relates to a pharmaceutical composition comprising a destruxin according to the invention together with a pharmaceutically acceptable excipient.

Preferably, said composition contains an effective amount of the compound according to the invention. Preferably, said composition is administered to a patient or animal in need thereof.

The present invention also relates to a destruxin according to the invention for the treatment and/or prevention of Clostridium perfringens-related bacterial infections, such as intestinal infections, including necrotic enteritis.

The pharmaceutical compositions according to the invention may be presented in forms for parenteral or oral administration.

They will therefore be presented in the form of injectable solutions or suspensions or multi-dose vials, as naked or coated tablets, dragees, capsules, pills, tablets, powders, suppositories or rectal capsules, solutions or suspensions.

Suitable excipients for such administration are cellulose or microcrystalline cellulose derivatives, alkaline earth carbonates, magnesium phosphate, starches, modified starches, lactose for solid forms.

For parenteral use, water, aqueous solutions, saline and isotonic solutions are the most convenient vehicles.

The dosage may vary within sizable limits (0.5 mg to 1000 mg) depending on the therapeutic indication and route of administration, as well as the age and weight of the subject.

According to another object, the present invention also relates to food compositions comprising a destruxin according to the invention. Said compositions are particularly suitable for feeding to farm animals such as pigs or poultry or any other farm animal susceptible to infection by Clostridium perfringens.

According to another object, the present invention also relates to the use of a destruxin as a feed additive for farm animals including pigs and poultry for the treatment and/or prevention of bacterial infections by the Clostridium perfringens strain.

The following examples illustrate, but do not limit, the invention. The starting products used are known products or are prepared according to known procedures.

### FIGURES

FIG. 1 illustrates the evaluation of resistance induction in Clostridium perfringens by destruxin A or B and metronidazole. The occurrence of resistant mutants was assessed in the presence of destruxin A or B or metronidazole as indicated in the text.

FIG. 2 illustrates the evaluation of the permeabilising effect of destruxins on Clostridium perfringens. Clostridium perfringens was exposed to destruxin A or B, nisin, enniatin A1 or CTAB at 5 times their MIC for 2 hours. The permeabilisation of the bacterial membrane was measured using propidium iodide as explained in the text. Permeabilisation is expressed as a percentage, with CTAB serving as a positive control and giving 100% permeabilisation. The values shown in the graph are the average +/− standard deviation.

FIG. 3 shows the determination of the critical insertion pressure of destruxins in a monolayer of lipids extracted from Clostridium perfringens. The critical insertion pressures of destruxins A and B, nisin, enniatin A1 and CTAB were measured as indicated in the text at a dose corresponding to 5 times their MIC.

FIG. 4 illustrates the determination of the insertion capacity of destruxins in a monolayer formed from lipids extracted from Clostridium perfringens and having an initial surface pressure corresponding to the membrane of the bacteria. The insertion of destruxins A and B, nisin, enniatin A1 and CTAB into a lipid monolayer mimicking the membrane of Clostridium perfringens was measured as indicated in the text at a dose corresponding to 5 times their MIC. The values shown in the graph are the average +/− standard deviation.

FIG. 5 shows the morphological phenotype of the bacterium Clostridium perfringens (ATCC 13124) incubated with different conventional antibiotics of known mechanism of action. Clostridium perfringens (ATCC 13124) was exposed to various conventional antibiotics acting on the synthesis of macromolecules indicated in the graph or to destruxin A (at a dose corresponding to 5 times their MIC). After 2 hours of exposure, the bacteria were labelled as indicated in the text before fluorescence microscopy of the phenotypes obtained.

### EXAMPLES

### I—Example 1: Evaluation of Antibacterial Activity

### Materials and Methods:

The antimicrobial activity of destruxins A and B was evaluated on the various commercial bacterial and fungal strains listed in Table 2 and obtained from ATCC, DSMZ or the Institut Pasteur (CIP). Antimicrobial activity was measured by determination of the Minimum Inhibitory Concentration or MIC according to the National Committee of Clinical Laboratory Standards (NCCLS, 1997) and as described in the following publications: Oyama et al, Nature Biofilms and Microbiomes, 2017, 3, 33; Benkhaled et al, Polym. Chem. 2018, 9, 3127-3141; Olleik et al, Eur J Med Chem, 2019, 165, 133-141.

The MIC is determined by exposing the bacteria or fungi to increasing doses of destruxin A or B or reference antibiotics obtained by serial dilution to ½ of these molecules in the culture medium.

Briefly, each bacterial or fungal strain was grown on a Petri dish containing the specific culture medium of the strain under study. One colony was collected and used to inoculate 3 mL of culture medium. After incubation at 37° C. under agitation (200 rotations per minute (rpm)) for 16 hours, the optical density (OD) was read at 600 nm to estimate the bacterial density. The bacterial suspension was then diluted 1:100 in 3 mL of culture medium before incubation at 37° C. under agitation at 200 rpm for 2-3 h until an OD₆₀₀ₙₘ of 0.6 was obtained.

The bacteria were then diluted to a density of 10^{E5} bacteria per milliliter (10^{E5} bacteria/mL). For fungal strains, the density used was 10^{E3} cells per mL for Candida albicans and 10^{E4} conidia per mil for the other fungi. 100 μL of this bacterial suspension was then added to wells of a 96-well polypropylene plate (Greiner BioOne) already containing 100 μL of destruxin A or B or reference antibiotics serially diluted to ½ in culture medium. The 96-well plates were then incubated under the temperature and time conditions indicated in Table 2, depending on the strain being tested.

For anaerobic strains (including all Clostridium strains), MIC was measured using an anaerobic chamber (Coy Laboratory Products, Grass Lake, Mich.).

For micro-anaerobic strains (H. pylori and E. faecalis) the MIC was measured using BD GasPack micro-anaerobic systems.

At the end of incubation, the OD₆₀₀ₙₘ was read using a microplate reader (Synergy Mx, Biotek), the MIC corresponding to the lowest concentration of destruxin A or B or reference antibiotic capable of inhibiting the increase in OD₆₀₀ₙₘ caused by bacterial or fungal growth. The test was repeated three times independently (n=3).

The Minimum Bactericidal Concentration (MBC), which is the lowest concentration that kills more than 99.9% of bacteria or fungi, was also measured. The MBC was measured by spreading 10 μL of the contents of the wells of the 96-well plates used in the MIC measurement on a Petri dish. After incubation under the strain-specific conditions listed in Table 2, the number of bacterial/fungal colonies was determined. Antibiotic concentrations giving a single colony or no colony were considered to be the MBCs.

Resistance induction has also been evaluated as described in the following publications: Oyama et al, Nature Biofilms and Microbiomes, 2017, 3, 33; Benkhaled et al, Polym. Chem., 2018, 9, 3127-3141. For this purpose, the bacterium Clostridium perfringens (ATCC13124) was exposed to destruxin A, destruxin B or metronidazole for 18 consecutive days. Each day the MIC was measured as described above. The last well where bacterial growth occurred (i.e. the MIC divided by two) was used to prepare the inoculum for measuring the MIC the following day.

TABLE 2
Strains tested and culture conditions used.
LB: Luria-Bertoni medium; MH: Mueller-Hinton medium; BHI: Brain Heart Infusion medium;
TS: Tryptocasein Soy medium; PD: Potato Dextrose; RPMI: Roswell Park Memorial Institute
medium; Middlebrook 7H9 and 7H10: selective medium for Mycobacterium.
[Table 1]

| | | | | Medium | T° of | Incu- |
|---|---|---|---|---|---|---|
| | | | Culture | used for | the | bation |
| | | Strain | medium | MIC test | MIC | on time |
| Gram (−) | Aerobic | Acinetobacter baumannii | LB | MH | 37 | 16-24 |
| | | (CIP 110431) | | | | |
| | | Citrobacter farmeri | LB | MH | 37 | 16-24 |
| | | (ATCC 51633) | | | | |
| | | Citrobacter rodentium | LB | MH | 37 | 16-24 |
| | | (ATCC 51116) | | | | |
| | | Escherichia coli (ATCC 8739) | LB | MH | 37 | 16-24 |
| | | Klebsiella pneumoniae | LB | MH | 37 | 16-24 |
| | | (DSM 26371) | | | | |
| | | Klebsiella variicola (DSM 15968) | LB | MH | 37 | 16-24 |
| | | Pseudomonas aeruginosa | LB | MH | 37 | 16-24 |
| | | (CIP 107398) | | | | |
| | | Salmonella enterica (CIP 80.39) | LB | MH | 37 | 16-24 |
| | | Shigella flexneri (ATCC 12022) | LB | MH | 37 | 16-24 |
| | Anaerobic | Bacteroides thetaioataomicron | BHI | BHI | 37 | 16-24 |
| | | (DSM 2255) | | | | |
| | | Helicobacter pylori | BHI | MH | 37 | 24 |
| | | (ATCC 43504) | | | | |
| Gram (+) | Aerobic | Arthrobacter gandavensis | LB | MH | 37 | 48 |
| | | (DSM 2447) | | | | |
| | | Bacillus subtilis (DSM 347) | LB | MH | 37 | 16-24 |
| | | Bacillus cereus (DSM 31) | LB | MH | 37 | 16-24 |
| | | Lactococcus lactis (DSM 20481) | LB | MH | 37 | 16-24 |
| | | Listeria monocytogenes | BHI | BHI | 37 | 16-24 |
| | | (DSM 20600) | | | | |
| | | Micrococcus luteus (DSM | TS | TSB | 37 | 16-24 |
| | | 20030) | | | | |
| | | Staphylococcus aureus MRSA | LB | MH | 37 | 16-24 |
| | | USA300 (ATCC BAA-1717) | | | | |
| | | Staphylococcus aureus (ATCC | LB | MH | 37 | 16-24 |
| | | 6538P) | | | | |
| | Anaerobic | Clostridium botulinum | BHI | BHI | 37 | 24-48 |
| | | (DSM 1985) | | | | |
| | | Clostridium coccoides | BHI | BHI | 37 | 24-48 |
| | | (DSM 935) | | | | |
| | | Clostridium difficile (DSM 1296) | BHI | BHI | 37 | 24-48 |
| | | Clostridium nexile (DSM 1787) | BHI | BHI | 37 | 24-48 |
| | | Clostridium perfringens | BHI | LB | 37 | 24-48 |
| | | (ATCC 13124) | | | | |
| | | Clostridium propionicum | BHI | BHI | 37 | 48-72 |
| | | (DSM 6251) | | | | |
| | | Enterococcus faecalis | LB | MH | 37 | 16-24 |
| | | (DSM 13591) | | | | |
| | | Lactobacillus acidophilus | BHI | BHI | 37 | 24-48 |
| | | (DSM 20079) | | | | |
| | | Propionibacterium acnes | BHI | BHI | 37 | 24-48 |
| | | (ATCC 6919) | | | | |
| | | Streptococcus pyogenes | BHI | BHI | 37 | 24-48 |
| | | (DSM 20565) | | | | |
| | | Streptococcus thermophilus | BHI | BHI | 37 | 24-48 |
| | | (ATCC LMD-9) | | | | |
| Mycobac- | Aerobic | Mycobacterium smegmatis | Middlebrook | Middlebrook | 37 | 48-72 |
| terium | | (ATCC 700084) | 7H10 | 7H9 | | |
| Fungi | Aerobic | Aspergillus flavus (DSM 1959) | PD | RMPI | RT | 48-72 |
| | | Aspergillus niger (ATCC 9142) | PD | RMPI | RT | 48 |
| | | Aspergillus ochraceus | PD | RMPI | RT | 48-72 |
| | | (DSM 824) | | | | |
| | | Candida albicans (DSM 10697) | PD | RMPI | 35 | 24 |
| | | Fusarium graminearum | PD | RMPI | RT | 48-72 |
| | | (DSM 1095) | | | | |
| | | Fusarium oxysporum | PD | RMPI | RT | 48-72 |
| | | (DSM 62316) | | | | |
| | | Fusarium verticillioides | PD | RMPI | RT | 48-72 |
| | | (DSM 62264) | | | | |
| | | Penicillium verrucosum | PD | RMPI | RT | 48-72 |
| | | (DSM 12639) | | | | |

### Results:

The antimicrobial activities of destruxin A, destruxin B and reference antibiotics used for comparison are given in Tables 3 to 6.

TABLE 3
MIC values of DesA, DesB, Bafilomycin A1 and Bafilomycin B obtained on various bacterial and
fungal strains tested. MICs are expressed in micromolar units or μM (micromole per litre).
[Table 2]

| | | | | | MIC | MIC |
|---|---|---|---|---|---|---|
| | | Strain | MIC DesA | MIC DesB | Bafilo A1 | Bafilo B |
| Gram (−) | Aerobic | Acinetobacter | >100 | >100 | >100 | >100 |
| | | baumannii | | | | |
| | | (CIP 110431) | | | | |
| | | Citrobacter farmeri | >100 | >100 | 100 | >100 |
| | | (ATCC 51633) | | | | |
| | | Citrobacter rodentium | >100 | >100 | >100 | >100 |
| | | (ATCC 51116) | | | | |
| | | Escherichia coli | >100 | >100 | 25 | >100 |
| | | (ATCC 8739) | | | | |
| | | Klebsiella pneumoniae | >100 | >100 | 100 | >100 |
| | | (DSMZ 26371) | | | | |
| | | Klebsiella variicola | >100 | >100 | >100 | >100 |
| | | (DSM 15968) | | | | |
| | | Pseudomonas | >100 | >100 | >100 | >100 |
| | | aeruginosa | | | | |
| | | (CIP 107398) | | | | |
| | | Salmonella enterica | >100 | >100 | >100 | >100 |
| | | (CIP 80.39) | | | | |
| | | Shigella flexneri | >100 | >100 | >100 | >100 |
| | | (ATCC 12022) | | | | |
| | Anaerobic | Bacteroides | >100 | >100 | >100 | >100 |
| | | thetaioataomicron | | | | |
| | | (DSM 2255) | | | | |
| | | Helicobacter pylori | >100 | >100 | >100 | >100 |
| | | (ATCC 43504) | | | | |
| Gram (+) | Aerobic | Arthrobacter | >100 | >100 | 3 | 3 |
| | | gandavensis | | | | |
| | | (DSM 2447) | | | | |
| | | Bacillus cereus | >100 | >100 | 12.5 | 25 |
| | | (DSM 31) | | | | |
| | | Bacillus subtilis | >100 | >100 | 100 | 20 |
| | | (DSM 347) | | | | |
| | | Lactococcus lactis | >100 | >100 | 50 | 50 |
| | | (DSM 20481) | | | | |
| | | Listeria monocytogenes | >100 | >100 | 25 | 50 |
| | | (DSM 20600) | | | | |
| | | Micrococcus luteus | >100 | >100 | 25 | 25 |
| | | (DSM 20030) | | | | |
| | | Staphylococcus aureus | >100 | >100 | >100 | 20 |
| | | MRSA USA300 | | | | |
| | | (ATCC BAA-1717) | | | | |
| | | Staphylococcus aureus | >100 | >100 | 12.5 | 10 |
| | | (ATCC 6538P) | | | | |
| | Anaerobic | Clostridium botulinum | >100 | >100 | 6.25 | 30 |
| | | (DSM 1985) | | | | |
| | | Clostridium coccoides | >100 | >100 | >100 | 30 |
| | | (DSM 935) | | | | |
| | | Clostridium difficile | >100 | >100 | >100 | >100 |
| | | (DSM 1296) | | | | |
| | | Clostridium nexile | >100 | >100 | 50 | 60 |
| | | (DSM 1787) | | | | |
| | | Clostridium perfringens | 1.5 | 3 | 25 | 10 |
| | | (ATCC 13124) | | | | |
| | | Clostridium | >100 | >100 | 25 | 25 |
| | | propionicum | | | | |
| | | (DSM 6251) | | | | |
| | | Enterococcus faecalis | >100 | >100 | >100 | 40 |
| | | (DSM 13591) | | | | |
| | | Lactobacillus | >100 | >100 | 33 | 10 |
| | | acidophilus | | | | |
| | | (DSM 20079) | | | | |
| | | Propionibacterium | >100 | >100 | 50 | 60 |
| | | acnes (ATCC 6919) | | | | |
| | | Streptococcus | >100 | >100 | 66 | 20 |
| | | pyogenes | | | | |
| | | (DSM 20565) | | | | |
| | | Streptococcus | >100 | >100 | 25 | 30 |
| | | thermophilus | | | | |
| | | (ATCC LMD-9) | | | | |
| Mycobac- | Aerobic | Mycobacterium | >100 | >100 | >100 | >100 |
| terium | | smegmatis | | | | |
| | | (ATCC 700084) | | | | |
| Fungi | Aerobic | Aspergillus flavus | >100 | >100 | 3.25 | 3.25 |
| | | (DSM 1959) | | | | |
| | | Aspergillus niger | >100 | >100 | >100 | 100 |
| | | (ATCC 9142) | | | | |
| | | Aspergillus ochraceus | >100 | >100 | >100 | 6.25 |
| | | (DSM 824) | | | | |
| | | Candida albicans | >100 | >100 | >100 | 100 |
| | | (DSM 10697) | | | | |
| | | Fusarium graminearum | >100 | >100 | 6.25 | 25 |
| | | (DSM 1095) | | | | |
| | | Fusarium oxysporum | >100 | >100 | >100 | 50 |
| | | (DSM 62316) | | | | |
| | | Fusarium verticillioides | >100 | >100 | 50 | 50 |
| | | (DSM 62264) | | | | |
| | | Penicillium verrucosum | >100 | >100 | 6.25 | 6.25 |
| | | (DSM 12639) | | | | |

The analysis of the results in Table 3 shows that:
Among all the bacterial and fungal strains tested, only the Clostridium perfringens strain (ATCC 13124) is sensitive to destruxin A and destruxin B with very good MIC values (1.5 and 3 μmol/L respectively) demonstrating the selectivity of destruxins A and B against the Clostridium perfringens strain (ATCC 13124).
Bafilomycin A1 and Bafilomycin B, two antibiotics with the same molecular target as destruxins in eukaryotic cells (the V-ATPase) are not selective for Clostridium perfringens and are thus active against many bacterial and fungal strains listed in Table 3.

TABLE 4
MIC values of DesA, DesB, Bafilomycin A1, B1 and metronidazole
obtained on various anaerobic bacterial strains. MICs are expressed in
micromolar units or μM (micromole per litre).

| | MIC | MIC | MIC | MIC | MIC |
|---|---|---|---|---|---|
| Strain | DesA | DesB | Bafilo A1 | Bafilo B | Metronidazole |
| Bacteroides | >100 | >100 | >100 | >100 | 2.34 |
| thetaioataomicron | | | | | |
| (DSM 2255) | | | | | |
| Clostridium | >100 | >100 | 6.25 | 30 | 0.29 |
| botulinum (DSM | | | | | |
| 1985) | | | | | |
| Clostridium | >100 | >100 | >100 | 30 | 0.29 |
| coccoides (DSM | | | | | |
| 935) | | | | | |
| Clostridium | >100 | >100 | >100 | >100 | 1.17 |
| difficile (DSM | | | | | |
| 1296) | | | | | |
| Clostridium nexile | >100 | >100 | 50 | 60 | 2.34 |
| (DSM 1787) | | | | | |
| Clostridium | 1.5 | 3 | 25 | 10 | 9.37 |
| perfringens (ATCC | | | | | |
| 13124) | | | | | |
| Clostridium | >100 | >100 | 25 | 25 | 1.17 |
| propionicum (DSM | | | | | |
| 6251) | | | | | |
| Enterococcus | >100 | >100 | >100 | 40 | >1500 |
| faecalis (DSM | | | | | |
| 13591) | | | | | |
| Helicobacter pylori | >100 | >100 | >100 | >100 | >1500 |
| (ATCC 21031) | | | | | |
| Lactobacillus | >100 | >100 | 33 | 10 | >1500 |
| acidophilus (DSM | | | | | |
| 20079) | | | | | |
| Propionibacterium | >100 | >100 | 50 | 60 | 1500 |
| acnes (ATCC 6919) | | | | | |
| Streptococcus | >100 | >100 | 66 | 20 | >1500 |
| pyogenes (DSM | | | | | |
| 20565) | | | | | |
| Streptococcus | >100 | >100 | 25 | 30 | 1500 |
| thermophilus | | | | | |
| (ATCC LMD-9) | | | | | |

The data in Table 4 confirm that:
Among all the anaerobic bacterial strains tested, only the Clostridium perfringens strain (ATCC 13124) is sensitive to destruxin A and destruxin B with very good MIC values (1.5 and 3 μmol/L respectively) demonstrating the selectivity of destruxins A and B against this strain.
Importantly, all other Clostridium strains tested (other than Clostridium perfringens) are insensitive to destruxins A and B (MIC>100 μM).
The data in Table 4 confirm that, unlike the destruxins, metronidazole, a conventional antibiotic active on many Gram+ and Gram− anaerobic bacteria, is not selective for Clostridium perfringens and is active on all tested Clostridium strains in Table 4.

TABLE 5
MIC values of various conventional antibiotics and destruxins
A and B against the strain Clostridium perfringens
(ATCC13124). MICs are expressed in micromolar units
or μM (micromole per litre) and mg/L (milligram per litre).

| Antibiotic | MIC (μM) | MIC (mg/L) |
|---|---|---|
| Bafilomycin A1 | 25 | 15.5 |
| Bafilomycin B1 | 10 | 8.15 |
| Chloramphenicol | 12.5 | 4.03 |
| Daptomycin | 8 | 12.96 |
| Destruxin A | 1.5 | 0.86 |
| Destruxin B | 3 | 1.78 |
| Enniatin A1 | 6.25 | 4.16 |
| Fosfomycin | 125 | 22.75 |
| Metronidazole | 9.37 | 1.60 |
| Tetracycline | 11.7 | 5.20 |

Analysis of the results in Table 5 shows that the MICs of destruxin A and destruxin B obtained on the Clostridium perfringens strain (ATCC 13124) are close to or lower than those of conventional antibiotics used to treat bacterial infections. Importantly, the MICs of destruxin A (1.5 μM or 0.86 mg/I) and destruxin B (3 μM or 1.78 mg/L) are significantly lower than that of metronidazole (9.37 μM or 1.6 mg/L), the conventional antibiotic commonly used in the treatment of intestinal infections caused by Clostridium perfringens.

TABLE 6
Comparison of MIC values of destruxins A and B and enniatin
A1 against various bacterial and fungal strains. MICs are expressed
in micromolar units or μM (micromole per litre).

| | Destruxin A | Destruxin B | Enniatin A1 |
|---|---|---|---|
| Bacillus subtilis (DSM | >100 | >100 | 25 |
| 347) | | | |
| Clostridium | 1.5 | 3 | 6.25 |
| perfringens (ATCC | | | |
| 13124) | | | |
| Enterococcus faecalis | >100 | >100 | 6.25 |
| (DSM 13591) | | | |
| Escherichia coli (ATCC | >100 | >100 | >100 |
| 8739) | | | |
| Pseudomonas | >100 | >100 | >100 |
| aeruginosa (CIP | | | |
| 107398) | | | |
| Staphylococcus | >100 | >100 | 25 |
| aureus (ATCC 6538P) | | | |
| Candida albicans | >100 | >100 | 3.125 |
| (DSM 10697) | | | |

The data in Table 6 show that enniatin A1, a fungal cyclic peptide belonging to the depsipeptide family like the destruxins, does not have the narrow selectivity of the destruxins against Clostridium perfringens since enniatin A1 is active against various Gram+ strains and the yeast Candida albicans as shown in Table 6. This demonstrates that the antimicrobial properties of destruxins, particularly their selectivity against Clostridium perfringens, are not shared by other members of the same peptide family, the fungal depsipeptides.

TABLE 7
MIC values of destruxin A and destruxin B against various clinical strains
of Clostridium perfringens isolated from human patients or animals.
MICs are expressed in micromolar units or μM (micromole per litre).

| Strain | MIC DesA | MIC DesB |
|---|---|---|
| Human 779269 | 0.75 | 3 |
| Human 779790 | 1.5 | 3 |
| Human 779794 | 1.5 | 3 |
| Human 779809 | 1.5 | 3 |
| Human 664408 | 1.5 | 3 |
| Human 256313 | 1.5 | 3 |
| Human 597867 | 1.5 | 3 |
| Human 22151 | 1.5 | 3 |
| Human 366478 | 1.5 | 3 |
| Human 96289 | 1.5 | 3 |
| Animal 24 | 0.75 | 3 |
| Animal 56 | 3 | 3 |
| Animal 60 | 1.5 | 1.5 |

Analysis of the results in Table 7 shows that destruxin A and destruxin B have MICs on clinical strains of Clostridium perfringens (isolated from infected patients or animals) close to or lower than those obtained on the Clostridium perfringens strain (ATCC 13124). Thus, destruxins A and B have the same activity, or are even more active on strains isolated from patients or animals than on the reference ATCC strain, demonstrating their possible use in the treatment of humans and animals infected with Clostridium perfringens or in the preventive treatment of Clostridium perfringens infections in farm animals including pigs and poultry.

The minimum bactericidal concentrations of destruxin A and destruxin B were determined on Clostridium perfringens strain (ATCC 13124) and on clinical strains of Clostridium perfringens isolated from patients or animals. In all cases, the BMC values are identical to the MIC values obtained, demonstrating that destruxins A and B have a bacteriolytic action on Clostridium perfringens.

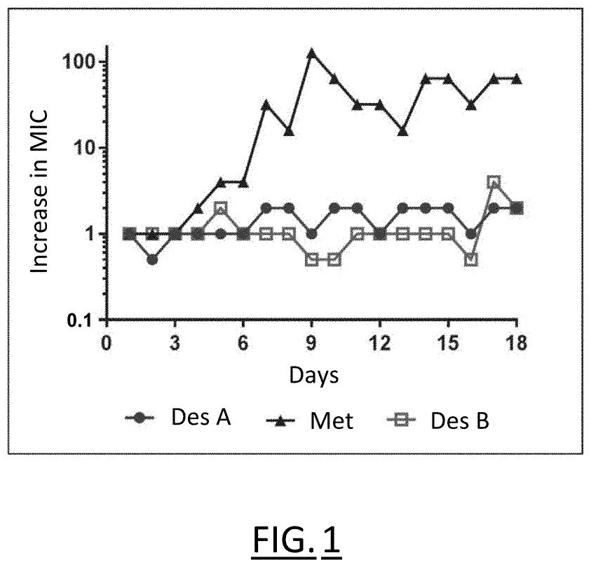

Most importantly, evaluation of resistance induction in Clostridium perfringens (ATCC 13124) exposed to destruxin A or B or metronidazole for 18 days showed that (FIG. 1):
the MICs of destruxin A and B do not change or change only slightly (with a doubling of the MIC value) during the 18 consecutive days of contact
Conversely, metronidazole rapidly leads to the development of resistance with a significant increase in its MIC: 10-fold increase in MIC after 7 days of contact and 50-100-fold increase after 9 days of contact.

This demonstrates that, unlike a conventional antibiotic such as metronidazole, destruxins A and B do not lead to the appearance and/or selection of mutants of Clostridium perfringens resistant to their action.

In conclusion, the antimicrobial activity tests of destruxins A and B show that:
In contrast to conventional antibiotics, including metronidazole, destruxins A and B are highly selective, showing activity only against commercial and clinical strains of Clostridium perfringens with a very low MIC/BMC (0.75-3 μM). This very narrow selectivity is a major advantage of destruxins because, unlike conventional antibiotics which strongly disturb the intestinal commensal flora, the use of destruxins will not lead to intestinal dysbiosis. This could also allow preventive treatment of livestock, including pigs and poultry, with destruxins to prevent Clostridium perfringens infection without the risk of disturbing the beneficial commensal flora of the animals.
The selectivity of destruxins A and B against Clostridium perfringens is not present in other members of the fungal depsipeptide family including enniatin A1 demonstrating that not all members of the depsipeptide family have the same antimicrobial activities.
Unlike conventional antibiotics, such as metronidazole, repeated exposure to destruxin A and B does not lead to the development of resistant strains. Furthermore, these results show that destruxins are active against the metronidazole-resistant strain of Clostridium perfringens (ATCC13124). These two observations reinforce the therapeutic and/or preventive interest of destruxins in the current context of a constant increase in the number of bacterial strains resistant or multi-resistant to conventional antibiotics.

### II—Example 2: Evaluation of the Safety and Transepithelial Passage of Destruxins A and B Using Human and Animal Intestinal Cells

### Materials and Methods:

The safety of dextruxins was first measured by a haemolysis test performed on human red blood cells as published in the paper Oyama et al, Nature Biofilms and Microbiomes, 2017, 3, 33. Red blood cells obtained from Divbioscience (Netherlands) were washed 3 times in phosphate buffer (PBS, pH 7) and then diluted to 8% (volume: volume) in PBS. 100 μL of this cell suspension was added to 96-well plates and then 100 μL of PBS containing increasing doses of destruxin A or B was added to each well. After 1 h incubation at 37° C., the plates were centrifuged at 800 g for 5 min. 100 μL of supernatant was then transferred to a new 96-well plate before reading the optical density at 405 nm. The percentage of haemolysis caused by destruxins was quantified using Triton-X100 (Sigma Aldrich) as a positive control giving 100% haemolysis.

The safety and intestinal absorption of destruxins was also assessed using human intestinal cells mimicking the small intestine (Caco-2 cells (ATCC HTB-37) or colon (T84 cells (ATCC CCL-248) and using porcine small intestine cells (DSM ACC701). The cells were grown normally in Dulbecco's Modified Eagle Medium supplemented with 10% (volume: volume) fetal calf serum. The cells were seeded in 25 cm2 flasks and maintained at 37° C. in a CO2 incubator with the medium being changed every two days and moving to the next step when cells reached 80-90% confluence.

For safety testing, Caco-2, T84 or IPEC-J2 cells were trypsinised and seeded in 96-well plates. Once confluent, the cells were exposed to increasing doses of destruxin A or B or other molecules for 48 hours. After 48 hours of incubation, cell viability was measured using the Sigma-Aldrich resazurin-based toxicity assay kit (ref. TOX8-1KT). After 4 hours of incubation with the kit reagent, cell viability was measured by reading the fluorescence of the wells (excitation at 530 nm/emission at 590 nm). The concentration causing 50% cell death (IC50) was calculated graphically using GraphPad® Prism 7 software. Statistical analysis of the data was carried out using the software's t-test and ANOVA test.

For intestinal absorption tests, Caco-2, T84 or IPEC-J2 cells were seeded on Greiner culture inserts of 1 cm2 diameter and 0.4 μm porosity. After 21 days of culture, the tightness of the intestinal epithelium was confirmed by measuring the transepithelial electrical resistance using a voltohmeter (Millipore EVOM). The transepithelial (apical to basal) passage and intracellular accumulation of the various antibiotics (destruxins A or B, bafilomycin A1, metronidazole, enniatin A1) were measured after addition to the apical compartment (corresponding to the intestinal lumen) of 100 μM of compound diluted in PBS containing calcium and magnesium (PBS++). After 4 hours of incubation at 37° C., the transepithelial electrical resistance was measured using a voltohmeter (Millipore EVOM) and the apical, basal and intracellular media were collected and analysed by HPLC chromatography to quantify the antibiotic content.

### Results:

The haemolysis test (Table 8) shows that destruxins A and B (such as bafilomycin A1 and metronidazole) do not induce any haemolysis, even at doses of 100 μM (0% haemolysis at 100 μM). Conversely, in accordance with its toxicity already known and reported in the literature, enniatin A1, which belongs to the same family of fungal cyclic peptides (depsipeptides), causes haemolysis of human red blood cells with 51% haemolysis observed at 100 μM enniatin A1.

Evaluation of the toxicity of destruxins A and B on human (Caco-2 and T84) and pig (IPEC-J2) intestinal cells (Table 9) shows that, like metronidazole, destruxins are not toxic even at 100 μM to these cells. Conversely, as reported in the literature, enniatin A1 is toxic to these intestinal cells. Similarly, bafilomycin A1 is toxic against all intestinal cells tested with IC50s between 1.8 and 11.8 μM.

TABLE 8
Evaluation of the haemolytic effect of destruxins A and B,
enniatin A1, bafilomycin A1 or metronidazole on human red blood
cells. The values in the table indicate the percentage of haemolysis
observed after 1 hour of contact with 100 μM of antibiotic.

| | Destruxin | Destruxin | Enniatin | Bafilomycin | Metronida- |
|---|---|---|---|---|---|
| | A | B | A1 | A1 | zole |
| Percentage | 0 +/− 0 | 0 +/− 0 | 51 +/− 12 | 0 +/− 0 | 0 +/− 0 |
| of | | | | | |
| haemolysis | | | | | |

TABLE 9
Evaluation of the toxicity of destruxins A and B, enniatin A1,
bafilomycin A1 or metronidazole on human and pig intestinal cells.
The values in the table correspond to the IC50 expressed
in μmol/L (μM) (mean +/− standard deviation).

| | Caco-2 | | |
|---|---|---|---|
| | (mimicking | T84 | IPEC-J2 |
| | the human | (mimicking | (mimicking |
| | small | the human | pig small |
| | intestine) | colon) | intestine) |
| Destruxin A | >100 | >100 | >100 |
| Destruxin B | >100 | >100 | >100 |
| Enniatin A1 | 3.1 +/− 0.5 | 4.5 +/− 1.2 | 5.1 +/− 0.6 |
| Bafilomycin | 11.8 +/− 2.0 | 9.9 +/− 0.9 | 1.8 +/− 1.9 |
| A1 | | | |
| Metronidazole | >1500 | >1500 | >1500 |

On the basis of the toxicity data, it can be concluded that, like metronidazole (the conventional reference antibiotic used to treat intestinal infections caused by Clostridium perfringens), destruxins A and B are not haemolytic and do not cause toxicity to human and pig cells at doses active against Clostridium perfringens. Based on the MIC and IC50 of destruxin A and B, the safety factor (calculated by dividing the IC50 obtained in the toxicity test by the MIC obtained with Clostridium perfringens (ATCC 13124)) is at least 66. Conversely, bafilomycin A1 and enniatin A1 show low-dose toxicity against human and pig cells (from 1.8 to 11.8 μM for bafilomycin A1 and from 3.1 to XX μM for enniatin A1). The MICs against Clostridium perfringens (ATCC 13124) being 25 μM for bafilomycin A1 and 6.25 μM for enniatin A1, unlike destruxins or metronidazole, these molecules do not have safety factors (safety factor less than 1 for bafilomycin A1 and for enniatin A1).

The lack of toxicity of destruxins A and B therefore also distinguishes them from enniatin A1, another fungal cyclic peptide of the depsipeptide type with antibacterial action, which is highly toxic.

Evaluation of the transepithelial transport and intracellular accumulation of destruxins A and B using human or porcine epithelial cells (Table 10) shows that intestinal absorption of destruxins A and B even after 4 h of incubation is low, ranging from 0.2 to 3.3% of the initial dose. Similarly, the intracellular accumulation of destruxins A and B after 4 h is low and ranges from 3.0 to 20.4% of the initial dose. This is in contrast to enniatin A1 and bafilomycin A1 which accumulate in the intestinal cells (19.5-77.4% of the initial dose) in connection with their high toxicity.

TABLE 10
Evaluation using human and porcine intestinal epithelial cells of the
transepithelial passage and intracellular accumulation of destruxins A and B,
enniatin A1, bafilomycin A1 and metronidazole.
The values shown in the table were measured by HPLC after 4 hours of
incubation with the cells and correspond to the percentage of the dose initially
added to the cells in the apical compartment.

| | | | | Bafilomycin | |
|---|---|---|---|---|---|
| | Destruxin A | Destruxin B | Enniatin A1 | A1 | Metronidazole |
| | Caco-2 (mimicking the human small intestine) | | | | |
| Apical | 88.3 | 92.4 | 58.7 | 0.1 | 80.2 |
| fraction | | | | | |
| Basal fraction | 2.3 | 2.5 | 1.95 | 22.5 | 12.9 |
| Intracellular | 9.2 | 5.5 | 43.2 | 77.4 | 12.7 |
| fraction | | | | | |
| | T84 (mimicking the human colon) | | | | |
| Apical | 87.6 | 81.6 | 80.5 | 2.5 | 71.3 |
| fraction | | | | | |
| Basal fraction | 3.3 | 2.0 | 2.0 | 30.5 | 9.7 |
| Intracellular | 15.1 | 20.4 | 19.5 | 68.0 | 19.0 |
| fraction | | | | | |
| | IPEC-J2 (mimicking pig small intestine) | | | | |
| Apical | 88.6 | 95.5 | 45.7 | 0.1 | 84.0 |
| fraction | | | | | |
| Basal fraction | 0.2 | 1.5 | 1.3 | 28.1 | 7.1 |
| Intracellular | 11.1 | 3.0 | 53.0 | 71.8 | 8.9 |
| fraction | | | | | |

Tissue integrity after 4 hours of exposure to destruxin A or B was assessed by measuring the transepithelial electrical resistance of human or porcine intestinal cells grown on inserts using an EVOM device (Table 11). The data show that destruxins, such as metronidazole, have little or no effect on transepithelial electrical resistance (decreases of between 2 and 17% for destruxins and between 0 and 34% for metronidazole) indicating that these molecules cause little or no damage to intestinal integrity. Conversely, enniatin A1 and bafilomycin A1 cause large decreases in transepithelial electrical resistance (decreases of 67-83% for enniatin A1 and 61-75% for bafilomycin A1), indicating significant damage to intestinal tissue integrity.

TABLE 11
Evaluation using human and porcine intestinal epithelial cells of the tissue damage
caused by destruxins A and B, enniatin A1, bafilomycin A1 and metronidazole.
Human and porcine intestinal cells grown on inserts were exposed for 4 hours to 100 μM of
each molecule added to the apical compartment of the inserts. After 4 hours, tissue integrity
was assessed by measuring the transepithelial electrical resistance. Values are in ohm.cm2
(mean +/− standard deviation).
[Table 12]

| | Control | Destruxin A | Destruxin B | Enniatin A1 | Bafilomycin A1 | Metronidazole |
|---|---|---|---|---|---|---|
| Caco-2 | 293 +/− | 281 +/− | 282 +/− | 95 +/− | 112 +/− | 252 +/− 13.7 |
| | 7.5 | 13.3 | 15.0 | 20.6 | 16.5 | |
| T84 | 449 +/− | 373 +/− | 377 +/− | 122 +/− | 118 +/− | 293 +/− 12.3 |
| | 35.2 | 21.7 | 10.4 | 11.3 | 10.4 | |
| IPEC-J2 | 786 +/− | 768 +/− | 763 +/− | 128 +/− | 191 +/− | 875 +/− 203 |
| | 12.5 | 41.3 | 21.2 | 3.2 | 62.2 | |

In conclusion, it appears that:
Destruxins A and B are non-haemolytic and have little or no toxicity to the human and porcine intestinal cells tested
Intracellular accumulation and transepithelial passage of destruxins A and B is low, which is good. Indeed, following their ingestion, destruxins A and B will remain mostly in the intestinal lumen which is an advantage for their use in the topical treatment and/or prevention of intestinal infections caused by Clostridium perfringens.

### III—Example 3: Identification of the Mechanism of Action of Destruxins A and B

### Materials and Methods:

The mechanism of action of destruxins A and B has been studied by various techniques. Most antimicrobial peptides (AMPs) reported in the literature are known to insert into the bacterial membrane forming pores and causing permeabilisation/lysis of the bacterial membrane. The ability of destruxins A and B to permeabilise the membrane of Clostridium perfringens (ATCC 13124) was therefore assessed. Nisin, an AMP known to permeabilise the bacterial membrane, was used as a positive permeabilisation control. The pore-forming ability of enniatin A1 was also evaluated. Permeabilisation is assessed using propidium iodide, a molecule that fluoresces once it comes into contact with DNA (Oyama et al., Nature Biofilms and Microbiomes, 2017, 3, 33). The bacterial membrane is impermeable to propidium iodide, so it can only come into contact with DNA if the membrane is permeabilised. The principle of the test is as follows. A liquid culture of Clostridium perfringens (ATCC 13124) is centrifuged at 3000 rpm for 5 min. The bacterial pellet is then resuspended in PBS at a concentration of 10^{E9} bacteria/mL. Propidium iodide (Sigma Aldrich) is then added to this bacterial suspension at a final concentration of 60 μM. 100 μL of this suspension is then added to wells of a black fluorescence 96-well plate (Greiner) containing 100 μL of test molecules diluted in PBS to a concentration corresponding to 5 times their MIC. After 120 min of incubation at 37° C. under anaerobic conditions, the fluorescence of the wells was read using a fluorescence microplate reader (excitation at 530 nm and emission at 590 nm). Permeabilisation of the bacterial membrane of Clostridium perfringens (ATCC 13124) was expressed as a percentage, with CTAB serving as a reference and giving 100% permeabilisation.

The ability of destruxins A and B to insert into Clostridium perfringens membrane lipids has also been studied using the lipid monolayer or Langmuir balance technique (Oyama et al., Nature Biofilms and Microbiomes, 2017, 3, 33). Total membrane lipids of Clostridium perfringens (ATCC 13124) were extracted using the Folch technique (Oyama et al., Nature Biofilms and Microbiomes, 2017, 3, 33). These lipids were deposited with a Hamilton syringe on the surface of a drop of PBS forming a lipid monolayer at the PBS-air interface. During the test, the temperature is maintained at 20+/−0.2° C. The lipid film formed compresses a probe positioned on the surface of the PBS causing an increase in surface pressure measured with a surface microtensiometer (μTROUGH SX, Kibron Inc). Lipids are added until the desired surface pressure is reached, called the initial surface pressure (Pi, unit mN/m). Once the surface pressure has stabilised, the antibiotics to be tested are injected into the PBS sub-phase using another Hamilton syringe. If the injected antibiotic is able to insert itself into the lipid film, the surface pressure increases until it reaches a maximum value corresponding to the maximum surface pressure (Pmax in mN/m). The change in surface area caused by the insertion of the tested antibiotic (deltaP in mN/m) is calculated by subtracting Pi from Pmax (DeltaP=Pmax−Pi). The affinity of an antibiotic for the lipid film is assessed by measuring the critical insertion pressure (Pc). Pc is the initial surface pressure that does not allow the antibiotic to be inserted. Thus, the higher the Pc value, the more the antibiotic can be inserted into the lipid film. Pc is determined graphically by measuring the DeltaP caused by the insertion of the antibiotic at different Pi values (approximately 10, 15, 20, 25 and 30 mN/m). After plotting the points obtained in a graph of the type DeltaP (span on the Y-axis) against Pi (span on the X-axis), Pc is determined from the equation of the line obtained when Y (DeltaP)=0. In some experiments, to mimic the physiological situation, the DeltaP caused by the insertion of the antibiotic into the lipid film is measured for a surface pressure Pi set at 30+/−0.5 mN/m, the lipid membrane pressure of a whole bacterium theoretically being of this order (Oyama et al., Nature Biofilms and Microbiomes, 2017, 3, 33).

The mechanism of action of destruxins A and B on Clostridium perfringens (ATCC 13124) was also studied by microscopy as previously explained (Nonejuie et al, PNAS Oct. 1, 2013 110 (40) 16169-16174). A bacterial suspension of Clostridium perfringens (ATCC 13124) was diluted 1:100 and grown at 37° C. under anaerobic conditions until an optical density at 600 nm of 0.2 was reached. The bacteria were then treated for 2 hours with destruxin A or B or with various conventional antibiotics with known molecular targets. The dose of antibiotic used corresponds to 5 times their MIC. After 2 hours incubation, the bacterial membrane was labelled for 10 min in ice with the red fluorescent molecule FM4-64FX (from ThermoFisher, used at 12 μg/mL) and the bacterial DNA with the blue fluorescent molecule DAPI (from Sigma Aldrich, used at 2 μg/mL). The bacteria were then centrifuged at 7,500 rpm for 30 sec and washed with PBS. The bacteria were then fixed with 4% paraformaldehyde solution for 15 min on ice before being centrifuged again and washed with PBS. Finally, the bacteria were added to Vectashield mounting fluid (Vector Laboratories) and deposited between slide and coverslip before observation under a confocal microscope using specific FM4-64FX and DAPI filters (Olympus, Rungis, France).

### Results:

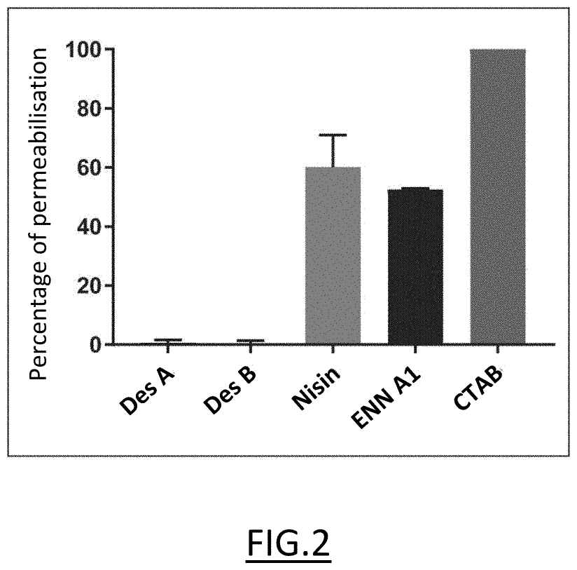

The permeabilising effect on Clostridium perfringens (ATCC 13124) of destruxins A and B was assessed using propidium iodide and is shown in FIG. 2. While nisin and enniantin A1 cause 50-60% permeabilisation of Clostridium perfringens (ATCC 13124), destruxins have no effect, causing no permeabilisation.

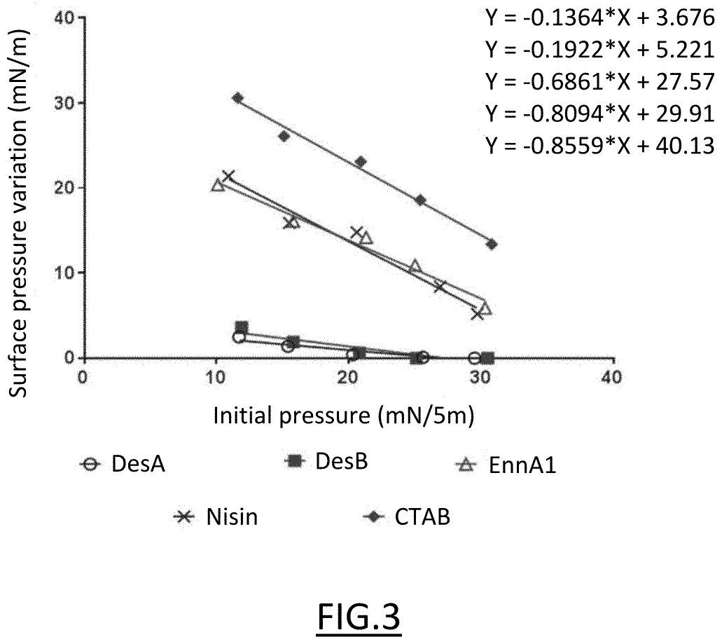

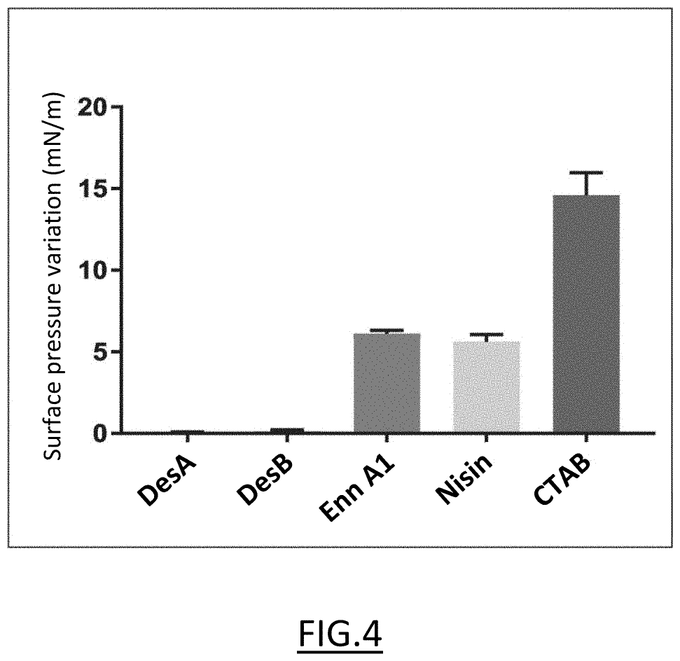

The lipid monolayer technique was then used to confirm the absence of insertion of destruxins A and B into the Clostridium perfringens membrane (FIGS. 3 and 4). Determination of the critical insertion pressure (Pc) (FIG. 3 and Table 12) shows that destruxins A and B insert very weakly into a lipid monolayer formed from total lipids extracted from Clostridium perfringens (ATCC 13124) with a Pc value of 26.9 and 27.1 mN/m for destruxin A and destruxin B. This means that destruxins A and B cannot be inserted into the Clostridium perfringens membrane with a theoretical surface pressure of 30 mN/m, as shown in FIG. 4 and in agreement with the permeabilisation data (FIG. 2). In contrast, enniatin A1, nisin and CTAB all have Pc values above 30 mN/m (40.1, 36.9 and 46.8 mN/m, respectively) indicating that they can therefore be inserted into a lipid monolayer formed from Clostridium perfringens lipids at the initial pressure of 30 mN/m (FIG. 4), consistent with their ability to permeabilise the bacteria (FIG. 2).

TABLE 12
Determination of the critical insertion pressure of destruxins A and B,
enniatin A1, nisin and CTAB.
The critical insertion pressures (Pc) of the different molecules injected
at 5 times their MIC were determined from FIG. 3.

| | Destruxin A | Destruxin B | Enniatin A1 | Nisin | CTAB |
|---|---|---|---|---|---|
| Pc (in mN/m) | 26.9 | 27.1 | 40.1 | 36.9 | 46.8 |

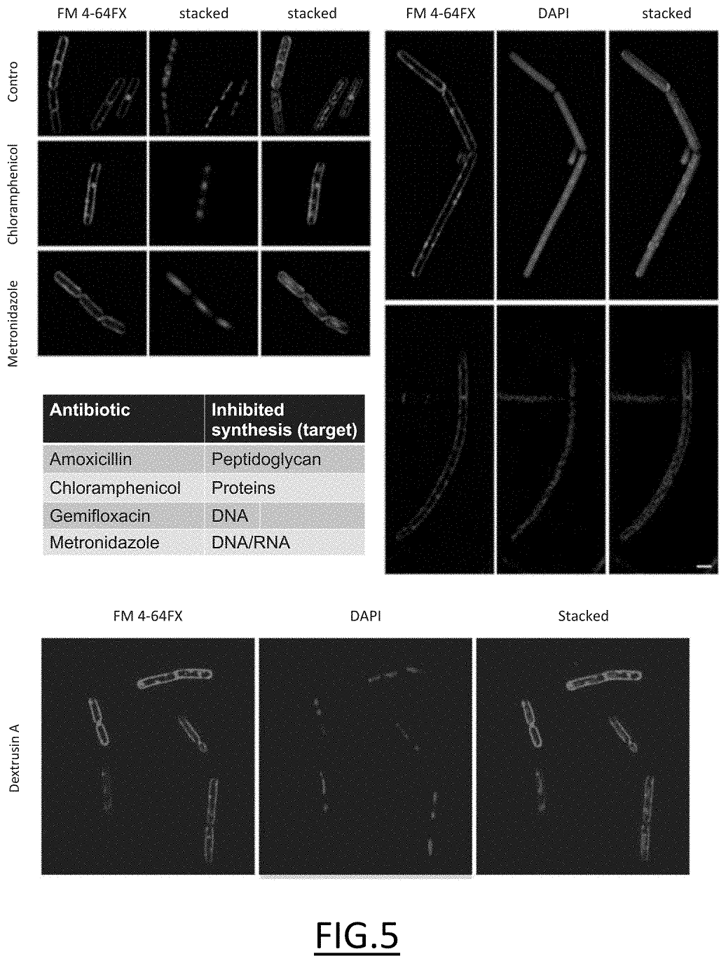

The results of the study of the mechanism of action of destruxins A and B on Clostridium perfringens (ATCC 13124) are shown in FIG. 5. Microscopy shows that destruxins act like chloramphenicol by causing hypercondensation of bacterial DNA. This suggests that, like chloramphenicol, destruxins act by inhibiting protein synthesis, an original mechanism of action for an AMP.

In conclusion:

Having shown the selective activity of destruxins A and B on Clostridium perfringens, the reasons for this selectivity were investigated. Classically, antimicrobial peptides (AMPs) have little or no selectivity, acting either on all Gram+ and Gram− bacteria, or only on Gram+ or Gram− bacteria, or on a set of phylogenetically related bacteria. Destruxins A and B are therefore very original since they only act on Clostridium perfringens (clinical and ATCC strains) without acting on phylogenetically related bacteria like the other Clostridium strains tested in Tables 3 and 4. The selectivity of destruxins A and B against Clostridium perfringens is due to their original mechanism of action.

Thus, while the majority of known AMPs act by permeabilising the bacterial membrane, destruxins A and B do not permeabilise the bacterial membrane of Clostridium perfringens (FIG. 2). Similarly, while the majority of AMPs are able to insert into bacterial lipids (activity monitored by the lipid monolayer technique), destruxins A and B do not penetrate a bacterial lipid film (FIGS. 3 and 4).

Microscopic tests were used to determine the mechanism of action of these molecules, which explains their specific action against bacteria of the genus Clostridium perfringens (FIG. 5). The existence of a specific target yet to be identified and present only in Clostridium perfringens certainly explains the selectivity of destruxins A and B against Clostridium perfringens. Importantly, the lack of membrane permeabilisation and lipid insertion of destruxins A and B distinguishes them from other fungal cyclic peptides with antibacterial action tested such as enniatin A1 which is able to insert into lipids and permeabilise the bacterial membrane (FIGS. 2, 3 and 4). This difference in mechanism of action between destruxins A and B and enniatin A1 explains the difference in selectivity of these molecules, a membrane permeabilisation type action being not or only slightly selective as shown by the non-selective action of enniatin A1 which is active against Clostridium perfringens but also against other Gram+ bacteria and against Candida albicans as illustrated in Table 6.

### IV—Example 4: Evaluation of the Antibacterial Activity and Safety of Supernatants (Secretomes) of Destruxin-Producing Fungi

### Materials and Methods:

In order to test the antimicrobial effects of destruxin-producing fungal extracts, the mould Beauveria felina (DSM 4678) was used. The secretomes of this mould were obtained after inoculation of Potato Dextrose (PD) medium with the fungus and cultured at 25° C. for 2 weeks under agitation (200 rpm). The secretomes obtained were centrifuged at 3000 rpm for 5 min and then sterilised by filtration through a 0.2 μm filter. The sterile secretomes were then used to perform antimicrobial testing as detailed in Example 1 by diluting 1:2 from the pure secretomes.

### Results:

The antimicrobial activity of Beauveria felina secretomes (DSM 4678) was evaluated on selected Gram+ and Gram− bacterial strains as shown in Table 13.

TABLE 13
Determination of the antimicrobial activity of the secretome obtained
from the mould Beauveria feline (DSM 4678).
The antimicrobial activity of Beauveria feline secretomes (DSM 4678)
was tested on various Gram+ and Gram− strains by serial dilution.
Activity is expressed as the percentage of diluted secretome with activity.

| | Percentage of lowest extract |
|---|---|
| | showing antimicrobial |
| Bacterial strains | activity against the tested strain |
| Clostridium perfringens (ATCC 13124) | 6.25% |
| Bacillus cereus (DSM 31) | 6.25% |
| Staphylococcus aureus (ATCC 6538P) | 12.5% |
| Escherichia coli (ATCC 8739) | 50% |
| Klebsiella pneumoniae (DSM 26371) | 50% |
| Pseudomonas aeruginosa (CIP 107398) | 50% |
| Salmonella enterica (CIP 80.39) | 50% |

The data in Table 13 shows that Beauveria felina (DSM 4678) secretomes are active against Clostridium perfringens (ATCC 13124) but also against other Gram+ pathogens such as Bacillus cereus (DSM 31) and Staphylococcus aureus (ATCC 6538P). This suggests that the secretomes contain other molecules, in addition to destruxins A and B, which are active against Gram+ bacteria. Although the loss of selectivity against Clostridium perfringens is detrimental, the fact that Beauveria felina (DSM 4678) secretomes are active against a variety of Gram+ pathogens infecting animals and humans is a positive finding. The use of more purified fractions of the secretome, enriched in destruxins, should make it possible to recover selectivity against Clostridium perfringens.

### SUMMARY OF FINDINGS

The above results therefore demonstrate the highly selective activity of destruxins A and B against bacteria of the genus Clostridium perfringens responsible for intestinal infections in humans and farm animals including pigs and poultry.

Due to the lack of toxicity of destruxins A and B at antibacterial doses, destruxins A and B can be used not only in the treatment of human Clostridium perfringens infections, but also in the treatment of intestinal Clostridium perfringens infection in farm animals, including pigs and poultry.

The fact that Beauveria felina (DSM 4678) secretomes are active against Clostridium perfringens makes it possible to propose the use of more or less purified extracts of this fungus or of other destruxin-producing fungi (such as Metarrhizium, Beauveria, Ophiocordyceps, Alternaria and Aschersoni and in particular the genera Metarrhizium anisopliae, Metarhizium brunneum, Beauveria felina, Ophiocordyceps coccidiicola, Alternaria brassicae, Alternaria linicola, Aschersonis sp, Ophiocordyceps coccidiicola, Alternaria brassice and Aschersonis sp) to prevent infection of livestock (including pigs and poultry) by Clostridium perfringens.

1. A composition comprising at least one destruxin for treating and/or preventing Clostridium perfringens infections.

2. The composition according to claim 1, such that the destruxin is selected from destruxins of the fungus series A, B, C, D, E, F or G, or derivatives thereof.

3. The composition according to claim 1 as having the general formula (I): 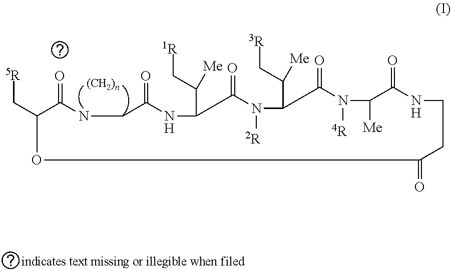
In which
¹R represents a hydrogen atom, a C1-C6 alkyl group or an aralkyl group;
²R, ³R, ⁴R, which may be identical or different, independently represent a hydrogen atom or a C1-C6 alkyl group;
⁵R represents a group selected from C2-C6 alkenyl and C1-C6 alkyl groups optionally substituted by one or more substituents selected from halogen atoms, hydroxy (OH), carboxy (COOH), -glycosyl groups, and 3- to 6-membered heterocyclic groups comprising one or more heteroatoms selected from N, O and S.

4. The composition according to claim 1, comprising a destruxin-producing fungus, or an extract thereof, such as a grind or culture supernatant thereof.

5. The composition according to claim 4 such that said fungus is selected from the genera Metarrhizium anisopliae, Metarhizium brunneum, Beauveria felina, Ophiocordyceps coccidiicola, Alternaria brassice, Alternaria linicola, Aschersonis sp, Ophiocordyceps coccidiicola, Alternaria brassice and Aschersonis sp or an extract thereof, and/or the culture supernatant thereof.

6. The composition according to claim 1 such that it is a feed supplement for farm animals including pigs and poultry.

7. A composition according to claim 1 such that it is a human or veterinary pharmaceutical composition for antibiotic use further comprising one or more pharmaceutically acceptable excipients.

8. A method for human or veterinary treatment of bacterial infections by the strain Clostridium perfringens comprising administering a destruxin or a destruxin-producing fungus, or an extract thereof, according to claim 1.

9. The method of claim 8 wherein destruxin has
the general formula (I): 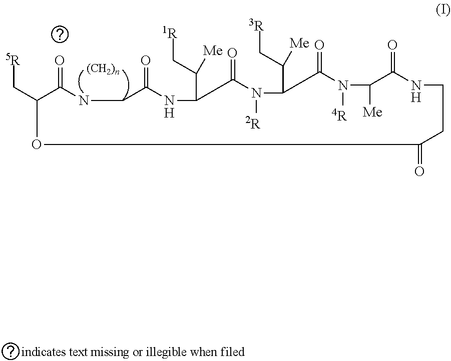
in which
¹R represents a hydrogen atom, a C1-C6 alkyl group or an aralkyl group;
²R, ³R, ⁴R, which may be identical or different, independently represent a hydrogen atom or a C1-C6 alkyl group;
⁵R represents a group selected from C2-C6 alkenyl and C1-C6 alkyl groups optionally substituted by one or more substituents selected from halogen atoms, hydroxy (OH), carboxy (COOH), -glycosyl groups, and 3- to 6-membered heterocyclic groups comprising one or more heteroatoms selected from N, O and S.

10. The method according to claim 8 where the infection is an intestinal infection such as necrotic enteritis.

11. Use of a destruxin or a destruxin-producing fungus, or an extract thereof as defined according to claim 1, as a feed additive for farm animals such as pigs and poultry, for the treatment and/or prevention of bacterial infections by the strain Clostridium perfringens.
